(19) United States
(12) Patent Application Publication
(10) Pub. No.: US 2003/0040676 A1
(43) Pub. Date: Feb. 27, 2003

(54) Method and apparatus for determining spatial relation of multiple implantable electrodes

(76) Inventors: John A. Prentice, Durango, CO (US); Jim Costello, Durango, CO (US); Mary Z. Fuka, Durango, CO (US)

Correspondence Address: GOTTLIEB RACKMAN & REISMAN PC, 270 MADISON AVENUE, 8TH FLOOR, NEW YORK, NY, 100160601

(21) Appl. No.: 10/132,862

(22) Filed: Apr. 25, 2002

Related U.S. Application Data
(60) Provisional application No. 60/288,358, filed on May 3, 2001.

(51) Int. Cl. A61B 5/04

(52) U.S. Cl. 600/508

(57) ABSTRACT

An apparatus and method to determine the relative locations of a set of implantable electrodes, preferably five or more, which can be positioned in a chamber of the heart or within another organ or chamber. At least two electrodes are configured to be a known distance from each other. A signal generation apparatus provides a signal having a frequency through a set of pairs of electrodes. This signal is detected on all other electrodes. Additional pairs of signal-emitting electrodes are selected and measurements are made on all other electrodes until sufficient sets of data have been acquired specify a set of equations. Solution of the sets of equations by numerical methods provides the relative locations of the electrodes in a dielectric medium. Where the electrodes are implanted in an environment comprising dielectric media of differing characteristics, such as blood and myocardial tissue, the signal generation apparatus is capable of producing signals at multiple frequencies. Sets of equations are acquired for multiple frequencies. Solutions of the equations derived from each of the sets of equations are combined to eliminate the effect of a non-uniform dielectric medium. Alternatively, the effect of differing media can be eliminated by calculating position vectors for image charges or virtual electrodes. Location of a dielectric boundary, such as the myocardial wall, may be determined.

This application claims the benefit of provisional application U.S. No. 60/288,358 filed May 3, 2001.

### BACKGROUND OF THE INVENTION

A. Field of Invention

This invention pertains to a method and apparatus for determining the three dimensional spatial relationship between multiple electrodes, more particularly, between electrodes implanted in the human body, and still more particularly between multiple electrodes implanted in the heart.

B. Description of the Prior Art

The heart is a mechanical pump that is stimulated by electrical impulses. The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV), which then pumps it throughout the body. Four heart valves keep the blood flowing in the proper directions.

The electrical signal that drives this mechanical contraction starts in the sino-atrial node, a collection of specialized heart cells in the right atrium that automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles, causing them to contract and pump blood to the lungs and body. This electrical activity occurs approximately seventy-two times a minute in a normal individual and is called normal sinus rhythm.

The corresponding electrical signals identifying these events are usually referred to as the P, QRS (or R) and T waves or beats. More particularly, an atrial contraction is represented on an ECG by a P wave, a ventricular contraction is represented by an R wave and a ventricular repolarization is represented by a T wave. The atrium also repolarizes but this event (the U wave) is masked by activity in the ventricle and consequently it is not observable on an ECG.

Electro-physiologic studies of the heart have been conducted using implantable multi-electrode catheters. The general location of those electrodes has frequently been determined by fluoroscopic inspection. An attending physician estimates the position of the electrodes by inspection of images. A system for determining electrode location is desirable, particularly where numerous electrodes are implanted.

In addition, proposals have been made for multi-electrode systems to sense, diagnose and treat cardiac conditions from bradycardia to tachyarrhythmias to fibrillation. Conventional pacing and defibrillation has relied on devices a limited number of electrodes implanted in the heart. For multi-electrode cardiac apparatus, identification of cardiac conditions and selection of appropriate therapies may rely, more or less, on the location of electrodes in the heart. Standard approaches for locating electrodes in the heart have relied on external imaging systems such as fluoroscopic images, external sensors, or electrode-lead geometry, for example, that the electrodes are distributed on a surface having a known shape or in a known spacing. Other methods require carefully controlled voltages applied to a reference electrode. The problem of locating a relatively large set of electrodes arbitrarily distributed in a bounded medium such as a chamber of the heart using only signals sent and received by those electrodes has not been solved in the art.

Other organs or body cavities may also be diagnosed or treated with catheters having multiple electrodes. It may also be advantageous to be able to locate the relative positions of the electrodes in space for diagnostic and treatment purposes.

### OBJECTIVES AND SUMMARY OF THE INVENTION

In view of the above art, it is an objective of the present invention to provide a method and apparatus for determining the relative locations of a system of electrodes in a dialectric medium. It is also an object of the invention to provide an apparatus which can determine the relative locations of a system of electrodes implanted in the human body. Such an apparatus may comprise an implantable cardiac stimulation system, such as a pacemaker, cardioverter, or defibrillator, in which five or more electrodes are positioned in a chamber of the heart.

A further objective is to provide an implantable system of electrodes wherein the relative position of five or more electrodes can determined in an organ or cavity of the human body. Another object of the invention is to provide an apparatus which can determine the relative location of a system of five or more electrodes in a dialectic medium. The technology developed may be useful in other applications.

Other objectives and advantages of the invention shall become apparent from the following description.

Briefly, the subject invention comprises a set of implantable electrodes, preferably five or more, which can be positioned in a chamber of the heart or within another organ or chamber. A method and apparatus are provided to determine the relative locations of the electrodes. Preferably two electrodes are configured to be a known distance from each other. A signal generation apparatus provides a signal having a frequency through a set of pairs of electrodes. This signal is detected on all other electrodes. Additional pairs of signal-emitting electrodes are selected and measurements are made on other electrodes until sufficient sets of data have been acquired specify a set of equations. Solution of the equations by numerical methods provides the relative locations of the electrodes in a dielectric medium.

Where the electrodes are implanted in an environment comprising dielectric media of differing characteristics, such as blood and myocardial tissue, the signal generation apparatus may be capable of producing signals at multiple frequencies. Sets of equations are acquired for multiple frequencies. Solutions of the equations derived from each of the sets of equations are combined to eliminate the effect of a non-uniform dielectric medium. In another embodiment, an algorithm compensates for the effects of boundaries by calculating fictitious point image charges or virtual electrodes.

A particular embodiment comprises an implantable cardiac stimulation system having a cardiac stimulator having electronic circuitry for the stimulation and a multi-electrode lead attached to the stimulator and inserted into one or more body cavities. (The term cardiac stimulator will be used herein to cover pacemakers as well as other cardiac devices such as internal cardioversion devices and defibrillators.) At least one lead having multiple electrodes is inserted into a cardiac cavity. Alternatively, the lead may be positioned in the veins, or it may be positioned externally of the heart. The implantable cardiac stimulation system may be adapted to sense intrinsic cardiac activity and to generate a stimulation pulse or pulses responsive to intrinsic cardiac activity, said stimulation pulse or pulses having an amplitude associated with a stimulation threshold.

In a preferred embodiment, a lead having an elongated member is provided with the electrodes being formed on said elongated member. The electrodes comprise axially spaced electrodes disposed on said elongated member, each electrode being connected by a wire extending though said elongated member. The electrodes may be circumferential coils integral or continuous with the wires or may be rings connected to the wires by crimping or laser welding, for example. An electrode may also be provided at the distal end of the lead. The elongated member may be a tube housing the wires. The electrodes can be angularly spaced with respect to each about the elongated member. The tube may include an elongated cavity adapted to receive a removable stylet. The stylet may be more rigid then the lead and may be used for the implantation of the lead. After the lead is implanted, the stylet is removed.

These and other features of the invention will be apparent from the following detailed description, taken with reference to the accompanying drawings.

### BRIEF DESCRIPTION OF THE DRAWINGS

FIG. 1 shows a diagrammatic front view of a patient with a cardiac stimulation system, including a programmer used to program the cardiac stimulator.

FIG. 2 shows a block diagram of the cardiac stimulator or FIG. 1.

FIG. 3 is a block diagram of a portion of the circuits of FIG. 2.

FIG. 4 is a second embodiment of the circuit portion of FIG. 3.

FIG. 5 is a block diagram of another portion of the circuits of FIG. 2.

FIG. 6 is a second embodiment of the other circuit portion of FIG. 5.

FIG. 7 is a block diagram of an adapter for connecting a multi-electrode lead to an IS-1 connector.

FIG. 8 is a view of a multi-electrode lead implanted in a heart.

FIG. 9 is a view of a second configuration of the multi-electrode lead in the heart.

FIG. 10 is a view of a third configuration of the multi-electrode lead in the heart.

FIG. 11 is a view of a fourth configuration of the multi-electrode lead in the heart.

FIG. 12 is a cross section of the multi-electrode lead of FIG. 8.

FIG. 13 is a flow chart for the development of a 3-D model of electrode position.

FIG. 14 is a graph of dielectric characteristics for blood and myocardium.

FIG. 15 is a flow chart for further development of the 3-D model of FIG. 13.

FIG. 16 is a flow chart for further development if the 3-D model of FIG. 13.

### DETAILED DESCRIPTION OF THE INVENTION

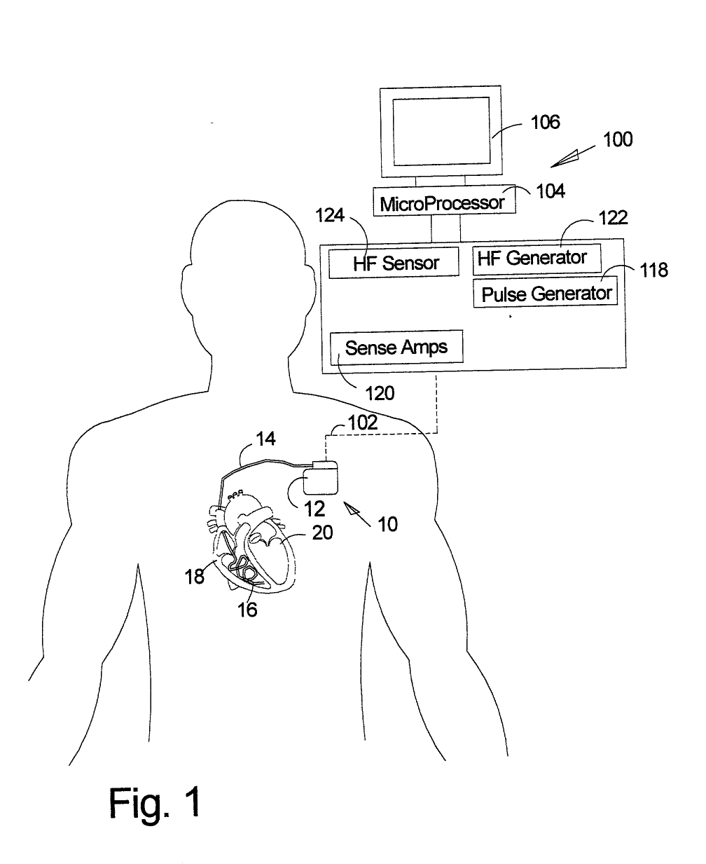

The subject invention may be employed in an implantable cardiac stimulation system 10 including a cardiac stimulator 12 with various electronic circuits, and a multi-electrode lead 14 attached to the stimulator 12, as shown. The lead 14 has a distal end 16 disposed, for example, in one of the cardiac chambers such as the right ventricle 18 of heart 20. In FIG. 1, end 16 is shown having a general spiral shape. The system 10 isadapted to deliver therapy in the form of electrical pulses. The therapy may include GCV (greater cardiac vein) resynchronization therapy, treatment of conduction pathway abnormalities, bardycardia pacing, cardioversion or defibrillation. The cardiac stimulator 12 contains electronic components common to current cardiac stimulators such as a battery, microprocessor control circuit, ROM, RAM, an oscillator, reed switch and antenna for communication, output circuits, and sense circuits. These components are well known to those of skill in the art. In addition the cardiac stimulator 12 has a plurality of independent sensing and stimulating circuits, as will be explained below.

Cardiac Stimulator

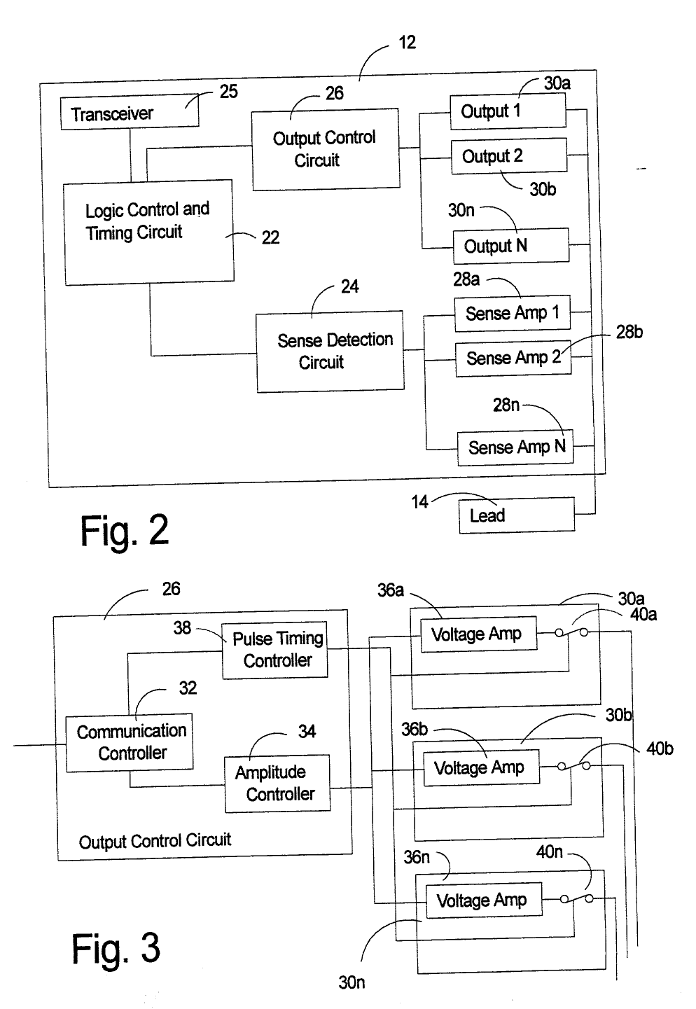

FIG. 2 illustrates important elements of the cardiac stimulator 12 in block diagram. The cardiac stimulator 12 comprises a logic control and timing circuit 22, which may include a microprocessor and memory, but which could also be implemented in a specialized circuit. The logic control and timing circuit 22 receives input from a sense detection circuit 24 and issues control instructions to an output control circuit 26. To accommodate the many electrodes used in the apparatus, multiple sense amplifiers 28a, 28b . . . 28n are provided, each in electrical communication with an electrode through the lead 14 and with the sense detection circuit 24. Similarly, the output control circuit 26 is electrically connected to a plurality of output circuits 30a, 30b . . . 30n. The output circuits 30a, 30b . . . 30n produce stimulating pulses or high frequency, non-simulating signals at electrodes in the heart through the lead 14. The logic control and timing circuit 22 may operate in accordance with a program stored into memory. The programming in memory is received through a transceiver 25 (for instance from programmer 100). During its operation, the microprocessor of the logic control and timing circuit 22 sets the output control circuit 26 and the sense detection circuit 24 in accordance with the appropriate electrode designations. Thereafter, the sensing detection circuit 24 senses intrinsic activity and other signals within the heart 20 and provides corresponding indication signals to the microprocessor. The logic control and timing circuit 22 then issues appropriate commands to the output control circuit 26. The output control circuit 26 generates appropriate stimulation pulses. These pulses are steered to the designated electrode or electrodes.

Output Circuits

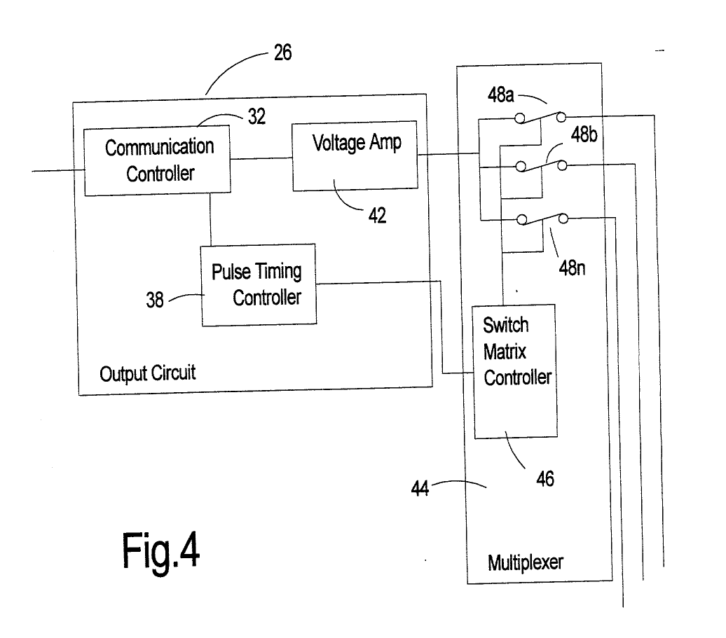

FIGS. 3 and 4 show two embodiments of output control circuits 26 and output circuits 30a, 30b . . . 30n. The embodiment of FIG. 3 comprises a communications controller that receives control signals from the logic control and timing circuit 22 (FIG. 2). Output of the communications controller 32 is sent to an amplitude controller 34 that controls the voltages produced by a plurality of voltage amplifiers 36a, 36b . . . 36n. In parallel, the communications controller 32 also regulates a pulse timing controller 38. Signals from the pulse timing controller 38 close and open switches 40a, 40b . . . 40n, thereby delivering stimulation pulses or high frequency signals to the heart through electrodes on the lead 14.

The embodiment of FIG. 4 also uses a communication controller 32 and pulse timing controller 38, but the amplitude controller 34 and plurality of voltage amplifiers 36a, 36b . . . 36n are replaced by a single voltage amplifier 42. To achieve the same effect of multiple pulses to selected electrodes, the signals from the pulse timing controller are sent to a multiplexer 44, comprising a switch matrix controller 46 and a plurality of switches 48a, 48b . . . 48n. The switches 48a, 48b . . . 48n may be opened and closed in a synchronized manner.

Sense Circuits

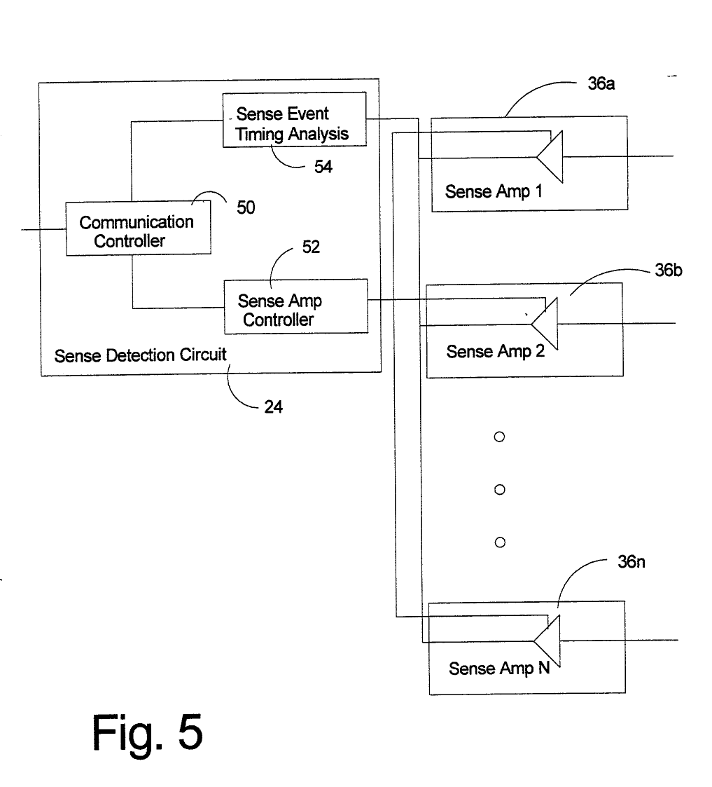

A variety of apparatus may also be used to sense signals from multiple electrodes through the sense detection circuit 24. A first embodiment is illustrated in FIG. 5. In the embodiment of FIG. 5, a communication controller 50 in the sense detection circuit 24 communicates with the logic control and timing circuit 22 (FIG. 2). The communication controller 50 is in electrical communication with a sense amp controller 52 and a sense event timing analysis unit 54. The sense amp controller 52 regulates amplification levels on the sense amps 36a, 36b . . . 36n such that significant signals are detected and noise is rejected. Each amplifier has independent sensitivity (gain) and filter characteristics. The sense event timing analysis unit 54 receives output from the sense amps 36a, 36b . . . 36n and collects that information. Information from high-frequency, non-stimulating signals is used to develop a representation of the location of the electrodes in three dimensional space, as described below.

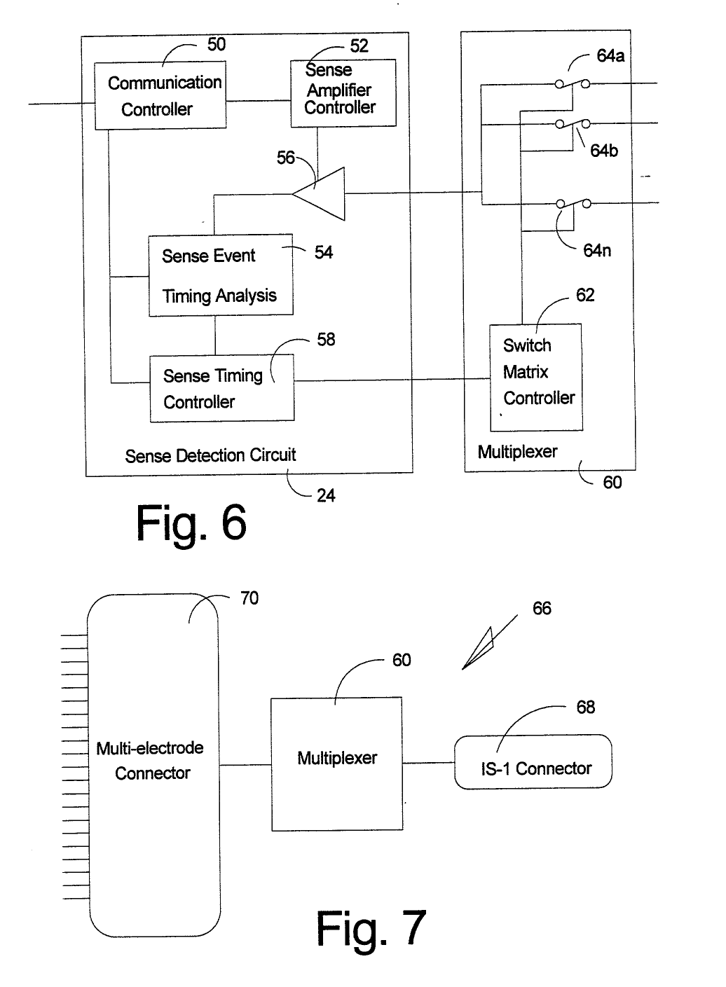

A second embodiment, illustrated in FIG. 6, employs a multiplexer in a manner similar to the second embodiment of the output control circuit, described in connection with FIG. 4, above. In this second embodiment of the sense detection circuit 24, the sense amp controller 52 controls a single amplifier 56. The sense event timing analysis unit 54 analyses the output of the single amplifier 56. A sense timing controller 58, in electrical communication with both the communication controller 50 and the sense event timing analysis unit 54, controls a multiplexer 60 through a switch matrix controller 62. The switch matrix controller 62 opens and closes a plurality of switches 64a, 64b . . . 64n, selectively connecting the electrodes of the lead 14 to the sense amplifier 56.

The multiplexers 44, 60 of the embodiments of the output control circuit of FIG. 4 and of the sense detection circuit of FIG. 6 may be combined externally to the cardiac stimulator 12 in an alternative configuration, illustrated in part in FIG. 7. FIG. 7 shows an adapter 66 for a connecting a multi-electrode lead to a cardiac stimulator having an IS-1 connector in the header of the stimulator 12. IS-1 connectors are well known and many physicians are familiar with their operation and use. For the adapter 68 a male IS-1 connector 68 is connected to the multiplexers 44, 60 in an independent package. The multiplexers are connected either directly to the lead 14 or indirectly through a multi-electrode connector 70. Dual chamber pacemakers having two IS-1 connectors in a single header are well known. In cardiac stimulators 12 according to the present invention using IS-1 connectors rather than a specialized multi-electrode connector, a first IS-1 connector might be used to carry both the voltage from the voltage amp 42 and signals from the pulse timing circuit 38 and a second IS-1 connector might be used to carry both the signals to the sense amplifier 56 and the control signals from the sense timing controller 58. Alternatively, one IS-1 connector might be dedicated to the control signals from the sense timing controller 58 and the pulse timing circuit 38 while another IS-1 connector might be dedicated to the signals delivered to and received from the heart, that is, to pulses from the voltage amp 42 and to sensed events.

Multi-Electrode Lead

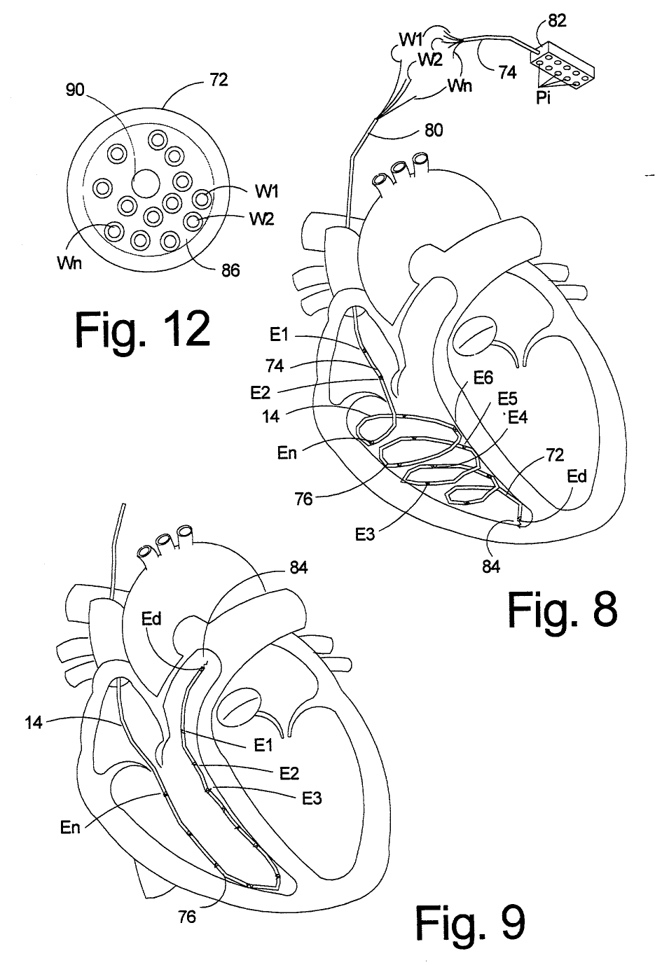

Details of the multi-electrode lead 14 are shown in FIG. 8. In this embodiment, the lead 14 includes an external biocompatible polymer tube 72 having a straight portion 74 and a shaped portion 76. The tube may be made of polyurethane or other similar materials that may be thermally shaped so that the shaped portion 76 retains any desired configuration. In FIGS. 1 and 8, the shaped portion 76 is shown as having a spiral shape, but many other shapes may be selected as well. The spiral or coil shaped lead of FIGS. 1 and 8 places electrodes around the entire chamber of the heart. This embodiment allows complete sensing and stimulating control around the entire chamber.

Another embodiment illustrated in FIG. 9 may provide a folded lead that places electrodes along the ventricular septum and up into the right ventricular outflow tract. This embodiment may be particularly useful where the applied therapy seeks to stiffen the septum, as further described below.

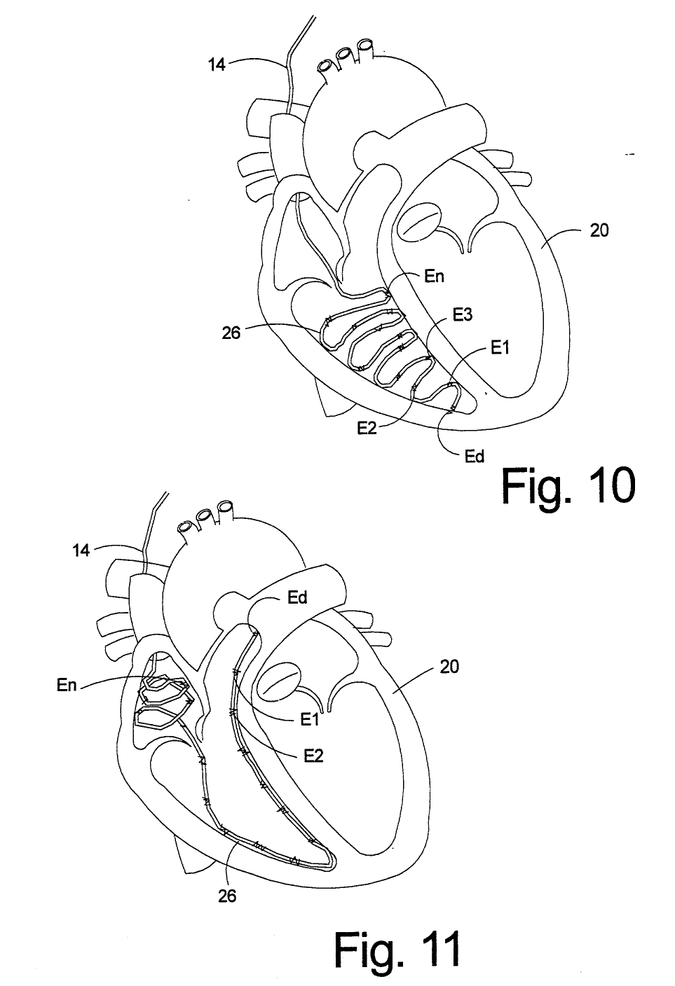

Yet another possible embodiment of FIG. 10 uses a serpentine shape to place electrodes along a wall of a chamber of the heart. These and other configurations may be combined and used in one or more chambers of the heart. FIG. 11, for example, shows a lead having a folded configuration in the right ventricle and a coiled or spiral configuration in the atrium. Such a configuration may have particular advantages for so-called single pass, dual chamber applications.

It will be apparent that numerous shapes could be selected to address the clinical needs of a particular patient. Moreover, the electrodes need not be mounted on a single lead. For purposes of this invention, it is sufficient that two electrodes be a known linear distance apart. This may be accomplished by designating two adjacent electrodes for this purpose, the two electrodes being separated by a relatively stiff segment of tube, or by a segment straightened, at least temporarily by a stylet. Alternatively, a relatively rigid, temporary lead with two electrodes held a known distance apart could be provided while the special locations of the other electrodes is determined, as explained in detail below.

Attached to tube 72 of the lead 14 of any configuration, there are provided a plurality of electrodes E1, E2, E3, E4, E5, . . . En. Preferably electrodes E1 . . . En are formed of coils of bare wire or cable wound about the tube 72. Each electrode is connected to corresponding wires W1, W2, W3 . . . Wn which extend through the length of tube 72 and which are shown exiting through end 80 for the sake of clarity. Wires W1, W2, W3 . . . Wn are insulated, so that they are not shorted to each other within the tube 72. The electrode 14 and its method of manufacture are disclosed in co-pending commonly assigned application Ser. No. 09/245,246 filed Feb. 5,1999 or application Ser. No. 09/761,333, and incorporated herein by reference. Preferably the end 80 of tube 72 and the ends of wires W1, W2, W3, etc. are coupled to a connector 82 for attaching the lead 14 to the cardiac stimulator 12. The connector 82 may have a plurality of pins Pi. Each wire W1 . . . Wn is associated with a pin. Because the lead may assume different configurations in the heart, it is the relative location of the electrodes in the heart that is important for application of an appropriate therapy, not the placement of the electrodes along the lead. In addition to spiral coil or ring electrodes E1 . . . En, a distal tip electrode Ed may also be provided. The distal tip electrode Ed may also have an active fixation mechanism, for example a helical screw 84 or tines, to secure the lead to the interior wall of the heart. The lead 14 can be constructed with the tube 72 extending relatively straight or can be customized to any shape to fit any pre-selected location within the heart 20 dependent on each particular patient's pathology.

The tube 72 can be formed with a longitudinal cavity 86, as shown in the cross sectional view of FIG. 12. Cavity 86 holds the wires W1, W2, W3 etc. The lead 14 could be straightened by inserting a substantially straight stylet 90 into cavity 86. The stylet 90 is also flexible but is less flexible than the lead 14 so that as it is inserted into the cavity 86, it forces the tube 72 to straighten. The lead 14 is then inserted into the heart or into a vein near the heart. After implantation of the lead 14, the stylet 90 is withdrawn and the lead 14 flexes back and takes a configuration shown, for example, in FIG. 8, 9, 10, or 11.

Programmer

A programmer 100 may be used to program the cardiac stimulator 12, usually by electromagnetic signals. In particular for use with this system, the programmer may be temporarily connected directly to the lead 14, as shown in FIG. 1 by dotted line 102. This connection may be made to the lead alone, or it may be made through the cardiac stimulator 12. Alternatively, if the invention is employed as an electrophysiology mapping catheter, the lead 14 or leads may be used solely with a programmer 100, without any implantable device 12. The connection to the programmer may be used after the lead has been implanted to characterize the location of the electrodes, as explained in detail below. The programmer 100 comprises a microprocessor 104 for performing various functions in connection with programming the cardiac stimulator or in connection with electro-physiologic tests. The programmer may also have sensing circuits 120 for sensing electrical events in the heart where a cardiac stimulator 12 is not used for this purpose. Finally, the programmer may have a high frequency generator 122 and high frequency sensor circuit 124, for providing non-stimulating high frequency signals that may be used to calculate the three dimensional positions of the electrodes within the patient's heart.

Electrode Identification

The process of identifying the optimum electrode or electrodes or a pattern of electrodes may be performed using several different approaches. For treatment of congestive heart failure, as well as for more traditional pacing modalities for bradycardia and tachycardia, the location of the electrode in the heart is important, not necessarily the position of any given electrode along the lead. As is apparent from FIGS. 8, 9, 10 and 11, an implanted lead may assume many configurations. The lead may overlap itself, whereby electrodes proximal on the lead are closer to the venticular apex than are more distal electrodes.

The relative position of the electrodes can be determined by measuring certain phenomenon and calculating a three dimensional position for each electrode, as described below. To determine the relative positions of the electrodes in three-dimensional space, calculations can be performed either in an external device such as the programmer 100, or in the cardiac stimulator 12. Because such calculations may be relatively energy expensive, calculation in an external device may be preferred. As described above, after implantation, the free end of lead 14 is connected to programmer 100, as shown in dotted line 102 in FIG. 1.

To apply the apparatus and method described herein to determine the location of electrodes in a dielectric medium, such as in blood within a chamber of the heart, it is necessary to establish a length scale for the system. This may be done by ensuring that two electrodes separated by a known distance, by utilizing the length of the catheter between distal and proximal electrodes, or by other means. Three non-colinear electrodes are selected to establish a reference system. The three non-colinear electrodes and the two electrodes separated by a known distance do not need to be the same. A set of at least five electrodes is needed, coupled to an alternating current voltage source. Only the frequency of the applied electrical signal must be known, as it is not necessary to control the voltage level of the applied signal quantitatively, provided the signal is stable.

As described above, the apparatus comprises the high frequency signal generator 122 which may be located in the external device, as illustrated, or in an implantable device. The range of frequencies will be discussed in further detail below. In addition there is the high frequency signal sensor 124, which may also be located in the external device, as illustrated, or in an implantable device. This sensor should be capable of measuring the voltage of the received signal at a sampling rate sufficiently high to satisfy the Nyquist sampling limit, that is, at a rate such that regularly spaced instantaneous samples of the waveform can completely determine the waveform of the signal. Preferably, this would comprise an analog to digital device connected to a digital computer, the A-to-D device sampling at a rate in excess of ten times the frequency of the signal generated by the high frequency signal generator 122.

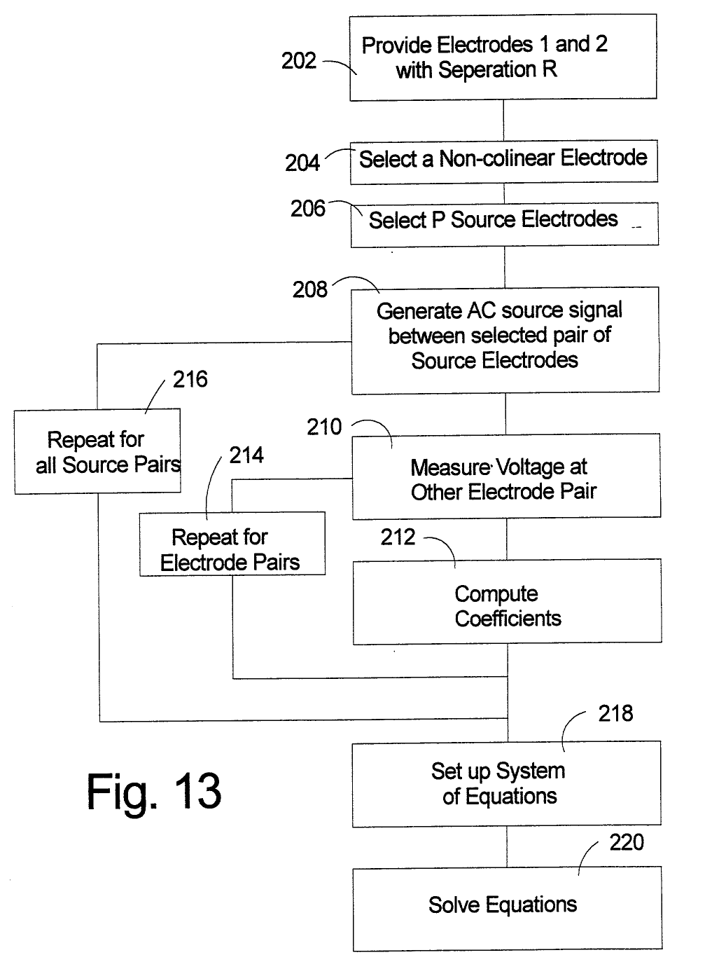

A general algorithm 200 for determining the relative three dimensional positions of a set of electrodes is illustrated in FIG. 13. At least five electrodes are implanted within a space such as a chamber of the heart. At least five electrodes are necessary to generate sufficient independent equations for a solution. Each electrode in the set is uniquely identified by number or in some other similar fashion such that the apparatus can distinguish between the electrodes. Two of the electrodes, preferably designated E1 and E2 and called the reference electrodes herein, may have a known separation R (step 202). For purposes of this algorithm, one of these two electrodes, for example E1, is designated as the origin of a Cartesean co-ordinate system having x, y, and z axis. The z axis is defined by the direction from E1 to E2, and the distance between E1 and E2 is used as a standard to calibrate other distances from the origin E1 to other electrodes. Each electrode will have a radius vector defining the electrode's location for this co-ordinate system. By definition, the radius vector r₁ for the electrode E1 is zero, since E1 is defined as the origin of the system. Similarly, the radius vector r₂ for the electrode E2 is defined as a vector having a length R equal to the known distance between E1 and E2 and a direction along the z axis with no components in an x or y direction.

A non-colinear third electrode is also selected at step 204. The third electrode is an electrode that is unlikely to be on a line connecting the reference electrodes E1 and E2. It need not be adjacent the reference electrodes. For example, in FIG. 8, if the reference electrodes are the electrodes designated E1 and E2, the third electrode might be electrode E6. In the configuration of FIG. 9, the third electrode might be the electrode designated En. The x axis is then defined as the projection of a position vector r_{w} for this third electrode into a plane perpendicular to the z axis. Therefore, the vector r_{w} will only have components in the x direction and in the z direction, that is, r_{w}=x_{w}^{Λ}x+z_{w}^{Λ}z, where ^{Λ}x and ^{Λ}z are unit vectors along the x and z axes respectively and x_{w} and z_{w} are magnitudes. The y axis is then mathematically defined as orthogonal to the previously defined z and x axes.

A set of P source electrodes is selected at step 206. In the preferred embodiment, all the electrodes are equally capable of emitting a high frequency signal and of receiving or detecting that signal. A signal would be emitted between a pair of source electrodes and detected between another pair of electrodes. However, in certain embodiments only some of the electrodes may be capable of emitting the high frequency signal. It may be desired to limit the quantity of data collected, to decrease processing time. In such circumstances, a minimum number of electrodes (P) must be capable of sending and receiving the high frequency signal. In order to generate sufficient independent equations, the total number of electrodes (N) must be at least five. If N is equal to 5, then P must also be 5, that is, all the electrodes must be capable of both sending and receiving. For N equal to 6 or 7, the number of sending electrodes P may be reduced to 3, and for N of 8 or greater, only 2 electrodes (P=2) are necessary. However, wherever possible, P should be made larger than the minimum number, as the additional information acquired will reduce error in the determination of the positions of the electrodes.

An AC source signal is generated between a selected pair of source electrodes at step 208. This series of steps will be performed for each unique pair of source electrodes, that is, (P(P−1))/2 times, as explained below. The signal is preferably a square wave, but any signal having sufficient power spectral density for at least one spectral component will do. It is desirable that the majority of the power spectral density for the selected signal be in the first harmonic term of the spectral decomposition. The frequency of the signal should be high enough such that no biological effects, such as contraction of the heart, are produced. In cardiologic applications, frequencies greater than 100 KHz do not ordinarily stimulate the heart to contract, but frequencies as low as 4 kHz can be used.

For the selected pair of source electrodes, the voltage and frequency of the supplied signal is measured at step 210 for each distinct pair of electrodes other than the selected source pair. This includes electrodes which will be or have been used as source electrodes. The voltage and frequency for each pair should be measured a number (M) of times (tₖ) during a time period when the signal is applied to the selected source electrodes. These measurements should occur in fixed relationship to the period of the signal, that is, measurements should be triggered at recurring points of the source signal. A number (m) of one or more time periods may also be used to acquire more data. For each sensing electrode pair, therefore, a set of voltage measurements is acquired. The number of measurements is equal to mM (number of time periods times the number of samples per period). This data may be acquired simultaneously on apparatus with multiple sense amplifiers (FIG. 2) or sequentially for multiplexed apparatus (FIG. 5).

For each set of voltages measured between a particular set of electrodes for a selected pair of source electrodes, a set of coefficients, Vmax, Y and Vdc, must be calculated at step 212. This is accomplished by minimizing the following sum:

$${\sum }_{k = 1}^{mM} {\vert V ( {t}_{k} ) - {V}_{max} sin ( ω {t}_{k} + θ ) - {V}_{D C} \vert}^{2}$$

This comprises a least-squares fit to the zero and first order Fourier components of the measured voltage. For further information on this method see Numerical Methods for Least Squares Problems, ke Björck, Society for Industrial and Applied Mathematics, 1996. Alternatively, the coefficients V^{max}, Θ and V^{DC} can be determined using digital signal processing techniques such as the discrete or fast Fourier transforms. See, for example, Discrete-Time Signal Processing, Alan V. Oppenheim, Ronald W. Shafer and John R. Buck, Prentice-Hall, 1999.

Next, as pointed out above, if data has not been acquired simultaneously on all electrode pairs, measurements must be repeated, through step 214, until sufficient unique, non-signal emitting pairs have been characterized by the coefficients mentioned above. For the N−2 electrodes not emitting a signal, one electrode may be a reference electrode and the remaining N−3 electrodes may be paired with the reference electrode for N−3 sets of coefficients. Every non-emitting electrode should be used in an electrode pair at least once. Therefore, minimally, N−3 pairs will be sampled.

When all of the coefficients have been determined for a selected pair of source electrodes, a new pair of source electrodes is selected at step 216, and the process of determining coefficients for electrode pairs of non-emitting electrodes is repeated until all the unique pairings of source electrodes have been selected. As pointed out above, this could be as few as one pair for a set of electrodes greater than eight, but could be as many as (N(N−1))/2, if all the available electrodes are used as both source and sensing electrodes.

After the sets of coefficients has been acquired for sufficient permutations of electrodes (every electrode used at least once) for permutations of selected source electrodes, a system of non-linear equations is set up at step 218. These equations can be solved by numerical methods. If the applied waveform was a square wave of frequency ω (radians per second), a system of non-linear equations can be generated in the form:

$${V}_{( i , j ) ( q , s )}^{D C} = Re [ {A}_{( i , j )} ] ( \frac{1}{\vert {r}_{q} - {r}_{i} \vert} + \frac{1}{\vert {r}_{q} - {r}_{j} \vert} - \frac{1}{\vert {r}_{s} - {r}_{i} \vert} - \frac{1}{\vert {r}_{s} - {r}_{j} \vert} )$$

$${V}_{( i , j ) , ( q , s )}^{max} = Re [ {B}_{( i , j )} ] ( \frac{1}{\vert {r}_{q} - {r}_{i} \vert} - \frac{1}{\vert {r}_{q} - {r}_{j} \vert} - \frac{1}{\vert {r}_{s} - {r}_{i} \vert} + \frac{1}{\vert {r}_{s} - {r}_{j} \vert} )$$

In the forgoing equations, i and j are indices for source electrodes, q and s are indices for the sensing electrodes, r is the position vector (in three dimensions) for the electrode identified by its subscript, A_{(i,j)} and B_{(i,j)} are complex-valued constants and Re is a function meaning to take the real value of the quantity in square brackets. This represents a system of (P(P−1)(N−3)) equations in 3N+2P−6 unknowns. It will be noted that, as explained above in connection with the reference electrodes, r_{w}=0, r₂=R^{Λ}z, and r_{w}=x_{w}^{Λ}x+z_{w}^{Λ}z. The condition r₂=R^{Λ}z, in particular, establishes the scale length for the system. The scale length of the system may be established in other ways. For example, if the distance between any two electrodes is known, the scale of the system may be established. Thus, two temporary electrodes on a separate, removable and more rigid catheter could be provided while the measurements and calculations are made. This would eliminate the need for a fixed linear distance on a lead that remains chronically implanted. Alternatively, after calculating proportional position vectors, without scaling, the scale could be established from three adjacent electrodes of known spacing on a flexible lead. Geometrically, these electrodes would be three points on an arc of known length and curvature. The cord or linear distance between any two electrodes can be calculated, thereby determining the scale of the system. Another alternative would be to estimate the scal from the approximate volume of the cardiac chamber.

With the set of equations established as above, the position vectors for the electrodes can be determined, step 220, by numerical methods. See, for example Numerical Methods for Least Squares Problems by ke Björck, Society for Industrial and Applied mathematics, 1996 or Numerical Optimization, Jorge Nocedal and Stephen J. Wright, Springer, 1999.

It is anticipated that the invention of this application will find particular, but not exclusive, application in connection with electrodes implanted in a chamber of the heart. Blood and myocardial tissue are both conducting dielectric materials. Their dielectric properties are represented by complex-valued dielectric permittivity. Since the blood and myocardium have different dielectric properties, additional modifications of the algorithm of FIG. 13 may be necessary under certain conditions. At frequencies below approximately 100 MHz, the dielectric properties of the blood and myocardium are different. As will be seen, there are at least three ways to utilize the characteristics of the blood and myocardium to minimize the effects of the myocardium on the algorithm described above.

Very High Frequency Sampling

The dielectric spectra of the blood and the myocardium are comprised of three main relaxation regions α, β, and γ at low medium and high frequencies. Numerous researchers have measured the dielectric spectrum of these materials as a function of frequency. See, for example, “The dielectric Properties of Biological Tissues: III. Parametic Models for the dielectric Spectrum of Tissues”, S. Gabriel, R. W. Lau, and C. Gabriel, Phys. Med. Biol. 41 (2996) 227-293. As shown in that paper, the dielectric spectrum can be characterized by a multiple Cole-Cole dispersion given by:

$$ϵ ( ω ) = {ϵ}_{∞} + {\sum }_{n} \frac{{Δϵ}_{n}}{1 + {( {ⅈωτ}_{n} )}^{( 1 - {a}_{n} )}} + \frac{{σ}_{I}}{{ⅈωϵ}_{0}}$$

where i=−1 and ε₀ is the permittivity of free space. Characteristic values of these various parameters for blood and myocardium are given by Gabrial, et al., op. cit., as:

| Parameter | Blood | Myocardium |
|---|---|---|
| ε | 4.0 | 4.0 |
| Δε₁ | 56.0 | 50.0 |
| T₁ (ps) | 8.38 | 7.96 |
| α₁ | 0.10 | 0.10 |
| Δε₂ | 5200 | 1200 |
| T₂ (ns) | 132.63 | 159.15 |
| α₂ | 0.10 | 0.05 |
| Δε₃ | 0.0 | 4.5 × 10⁵ |
| T₃ (μs) | — | 72.34 |
| α₃ | — | 0.22 |
| Δε₄ | 0.0 | 2.5 × 10⁷ |
| T₄ (ms) | — | 4.547 |
| α₄ | — | 0.0500 |
| σ₁ | 0.7000 | 0.0500 |

The real component of the inverse of the complex permittivity determines the electromagnetic field in a conducting dielectric medium, i.e., Re[1/ε(ω)]. FIG. 14 is a graph of the real component of 1/ε(ω) for blood and for myocardium as a function of frequency. The graph is derived from the above equation and the values given in the table. The curves converge toward a common value as the frequency of the applied field is increased. At frequencies of approximately 100 MHz and greater, 1/ε(ω) is essentially the same for both media and electromagnetic effect due to the myocardial boundary disappear. Thus if the High Frequency generator 122 is capable of developing frequencies in excess of 100 MHz, one need only selected the highest practicable frequency and apply the algorithm of FIG. 13. The effects of differences in materials can be eliminated thereby.

Projection to Very High Frequency

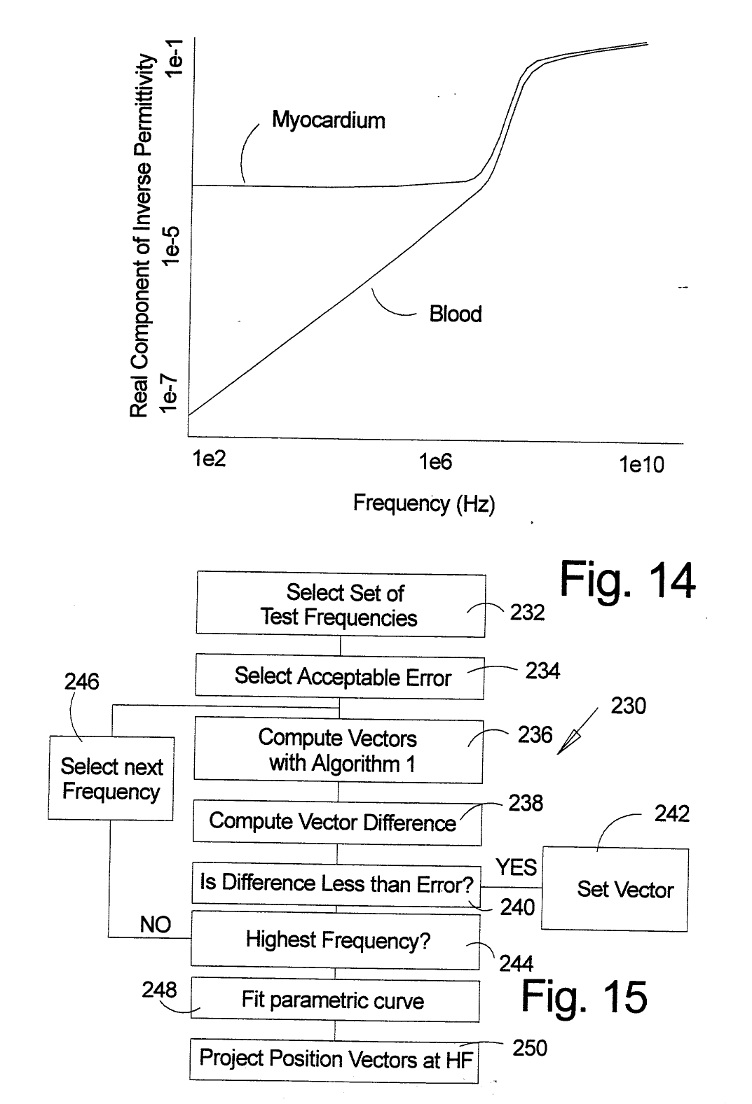

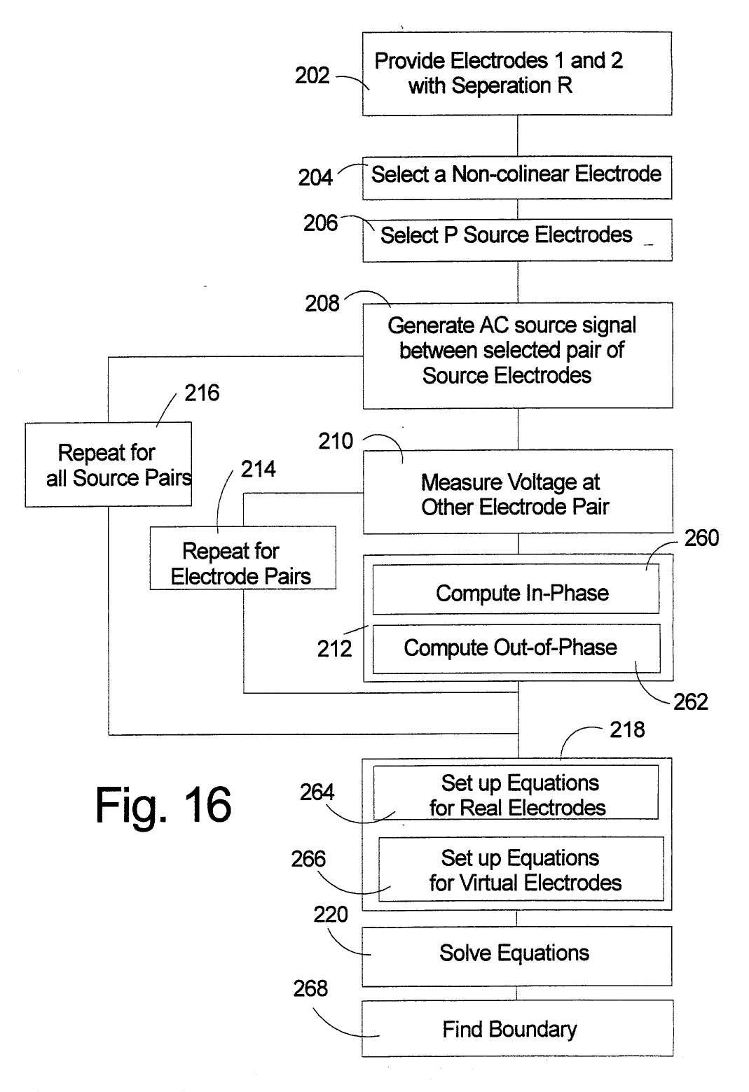

If, on the other hand, only lower frequencies are available, sampling at multiple frequencies can be used to project into the region where boundary effects can be ignored, as set forth in connection with the algorithm 230 of FIG. 15. If the High Frequency generator 122 is incapable of generating frequencies above 100 MHz, a set of test frequencies is selected at step 232. The frequencies should be of increasing value and should extend over a range wherein the value of 1/ε(ω) for blood is expected to increase, for example over a range from 100 Hz to 10 MHz. An acceptable error value is selected at step 234. This value represents the largest acceptable error for the determination of the position of the electrodes and will vary from application to application, but should usually be small compared to the expected distance between electrodes.

Beginning with an initial low frequency, the algorithm of FIG. 13 is utilized at step 236 to determine an initial value for each of the position vectors for the electrodes, as described above. At step 236, a vector difference between the presently calculated values for the position vectors and a set of previously calculated values. The vector difference is compared to the pre-selected error at step 240. Since there is no approximation for the location of the electrodes on the first iteration, the algorithm of FIG. 13 will be performed at least twice, at two different frequencies. If on the second or subsequent cycles, the difference at step 240 is less than the error, the final iteration is saved at step 242 as the representation of the positions of the electrodes.

If the vector difference is not less than the acceptable error, it is determined at step 244 if the highest available frequency has been selected. If a higher frequency is available, that frequency is selected (step 246). If, however, the highest available frequency has already been attempted, it is necessary to fit a parametric curve to the available data (step 248). The set of vectors acquired at each frequency are utilized to derive an equation as a function of frequency. Values are available for each electrode, and a separate curve is preferably fitted for each electrode. Techniques for computing parametric curves are known, for example, a least-squares fit to a polynominal. See, for example, Numerical Recipes in Fortran 77: The Art of Scientific Computing, William H. Press, Saul A. Teukolsky, William T. Vetterling, and Brian P. Flannery, Cambridge University Press, 2^{nd} Ed., 1992.

The fitted equation is then evaluated (step 250), at a frequency where 1/ε(ω) for blood and myocardium are expected to be nearly equal. For example, if the highest available frequency was below 100 MHz, the values for the position vectors should be derived from the equations using a frequency of approximately 500 MHz. If the highest frequency actually used was at or above 100 MHz, the equations may be evaluated at a much higher frequency. The resulting values for the position vectors are accepted and used in connection with other applications such as cardiac mapping, diagnostics, and therapy. The values may be computed in an external device and transmitted into an implantable device.

It will be noted that the algorithm of FIG. 15 can also be employed where higher frequencies are available form the high frequency generator 122, that is, frequencies above 100 MHz. This may improve the accuracy of the calculated position vectors even at the higher frequencies. For such application, frequencies should be chosen over as wide a range as possible. In either case, it may be advantageous to select the frequencies at constant intervals on a logarithmic scale.

Fictitious Image Charges or Virtual Electrodes

The algorithm of FIG. 13 may also be enhanced to account for the effects of the myocardium by modeling the effects of the dielectric boundary as if those effects by electrodes rather than by the wall of the heart. These fictitious point image charges or virtual electrodes are assumed to be across the heart wall from the real electrodes. In other words, for each real electrode, the effects of the heart wall are represented by a virtual electrode that is imagined to be the mirror image of the real electrode through a plane of the heart wall closest to the real electrode. The virtual electrode is therefore usually in the myocardium. The method of image charges is discussed generally in, for example, Classical Electrodynamics, John David Jackson, John Wiley & Sons, 1999. In the case of cardiovascular leads, it is expected that most real electrodes will be very close to the wall of the heart. The heart wall or dielectric boundary can be treated as an infinite plane in that circumstance.

To compensate for the effects of the myocardium, voltage measurements for the real electrodes are acquired as in the algorithm of FIG. 13. However, the coefficients are computed (step 212) to include both an in-phase (sin) term (step 260) and an out-of-phase (cos) term (step 262). The out-of-phase term represents the reflection of the signal from the heart wall, a reflection that is represented computationally by a virtual electrode. For each measured voltage V(tₖ) the quantities V^{max1}, V^{max2}, and V^{DC} are computed, as explained above, by minimizing the sum:

$${\sum }_{k = 1}^{mM} {\vert V ( {t}_{k} ) - {V}^{max 1} cos ( ω {t}_{k} ) - {V}^{max 2} sin ( ω {t}_{k} ) - {V}^{D C} \vert}^{2}$$

As before, this constitutes a least-squares fit to the zero and first order Fourier decomposition of the measured voltage. Other digital signal processing techniques such as discrete or fast Fourier transforms may also be used.

After the coefficients have been computed (step 212), the system of equations (step 218) must also be modified to use both the in-phase and out-of-phase coefficients and to represent both the real electrodes (step 264) and the virtual electrodes or image charges (step 266). The form of the equations for the system of non-linear equations takes the following forms:

$${V}_{( i , j ) ( q , s )}^{D C} = {A}_{( i , J )} ( \frac{1}{\vert {r}_{q} - {r}_{i} \vert} + \frac{1}{\vert {r}_{q} - {r}_{j} \vert} - \frac{1}{\vert {r}_{s} - {r}_{i} \vert} - \frac{1}{\vert {r}_{s} - {r}_{j} \vert} + {γ}_{( i , j )} [ \frac{1}{\vert {r}_{q} - {r}_{i}^{I} \vert} + \frac{1}{\vert {r}_{q} - {r}_{j}^{I} \vert} - \frac{1}{\vert {r}_{s} - {r}_{i}^{I} \vert} - \frac{1}{\vert {r}_{s} - {r}_{j}^{I} \vert} ] )$$

$${V}_{( i , j ) ( q , s )}^{max 1} = {B}_{( i , j )} ( \frac{1}{\vert {r}_{q} - {r}_{i}^{I} \vert} - \frac{1}{\vert {r}_{q} - {r}_{j}^{I} \vert} - \frac{1}{\vert {r}_{s} - {r}_{i}^{I} \vert} + \frac{1}{\vert {r}_{s} - {r}_{j}^{I} \vert} )$$

$${V}_{( i , j ) ( q , s )}^{max 2} = {C}_{( i , J )} ( \frac{1}{\vert {r}_{q} - {r}_{i} \vert} - \frac{1}{\vert {r}_{q} - {r}_{j} \vert} - \frac{1}{\vert {r}_{s} - {r}_{i} \vert} + \frac{1}{\vert {r}_{s} - {r}_{j} \vert} + {α}_{( i , j )} [ \frac{1}{\vert {r}_{q} - {r}_{i}^{I} \vert} + \frac{1}{\vert {r}_{q} - {r}_{j}^{I} \vert} - \frac{1}{\vert {r}_{s} - {r}_{i}^{I} \vert} + \frac{1}{\vert {r}_{s} - {r}_{j}^{I} \vert} ] )$$

In the foregoing equations, i and j are indices for source electrodes, q and s are indices for sensing (non-emitting) electrodes, A, B, C, y, and α are real-valued constants, r terms are position vectors for the electrodes indicated by the indices, and r^{I} terms are position vectors for the virtual electrodes or image charges associated with the real electrodes indicated by the indices. As explained above, this system of equations can be solved by various numerical methods implemented on a computer. Because there are additional unknown constants and additional position vectors for each of the virtual electrodes, more measurements must be made and more sets of coefficients must be determined in order to have a sufficiently large set of equations to determine all of the unknowns.

Moreover, in connection with cardiovascular applications, it may be observed that the electrical activity of the heart is effectively an additional direct current component which is not attributable to the signal produced on the source electrodes. The frequency of the signal produced at the source electrodes is high compared to the rate of contraction of the heart and the heart's associated electrical condition. The contribution of the heart's natural activity to V^{DC} cannot be easily distinguished from direct current effects associated with the signal from the source electrodes. It is preferred, therefore, to establish systems of equations using measured values for V^{max1}, V^{max2}, or V^{max} and not V^{DC}. This requires that the minimum number of samples from different electrode pairs be increased to obtain a sufficient set of equations. Of course, as mentioned above, exceeding the minimum number of equations by measuring between more different pairs of electrodes and calculating additional sets of coefficients improves accuracy and is therefore preferred.

The method of fictitious image charges or virtual electrodes provides additional useful information about the cardiac chamber in which the electrodes are implanted. By taking the vector average of the position vector for a real electrode and the position vector for its associated virtual electrode, an estimate of the position of the myocardial wall can be obtained (step 268). The position vector for the myocardial wall nearest an electrode is given by:

$${r}_{i}^{wall} = \frac{{r}_{i} + {r}_{i}^{I}}{2}$$

The method described herein may be extended from monopoles (that is, single image charges) to dipoles, quadrapoles and so on without departing from the teachings hereof. The addition of higher order will significantly increase the number of unknowns and make it necessary to increase both the minimum number of electrodes and the minimum number of source electrodes.

Numerous other modifications may be made to this invention without departing from its scope as defined in the attached claims.

What is claimed is:

1. A method for determining a spatial relationship between a plurality of electrodes implanted in a human body comprising
placing a set of at least five electrodes in a cavity in the body;
designating a first electrode as an origin;
designating a second electrode defining a first axis,
selecting a third electrode, said third electrode being non-colinear with said first and second electrodes, said third electrode defining a second axis,
defining a third axis related to said first and second axes;
generating a periodic signal having a frequency between two signal-generating electrodes,
measuring said signal at pairs of electrodes taken from said set of electrodes, excluding said two signal generating electrodes; and
determining a location vector for each electrode from said measured signals.

2. The method of claim 1 wherein said first and second electrodes are separated by a known distance.

3. The method of claim 1 wherein measuring said signal at pairs of electrodes further comprises measuring at every electrode at least once.

4. The method of claim 1 wherein determining a location vector for each electrode further comprises determining a location vector for each real electrode and determining a location vector for a virtual electrode associated with a real electrode.

5. The method of claim 4 further comprising determining the location of a dielectric boundary between a real electrode and a virtual electrode associated with said real electrode.

6. The method of claim 1 wherein said step of measuring comprises acquiring a plurality of sample measurements for each unique pair during a period of time when said signal is generated between said signal-generating electrodes.

7. The method of claim 6 wherein said step of determining a location vector comprises solving a set of non-linear equations.

8. The method of claim 6 wherein the step of determining a location vector further comprises fitting said plurality of sample measurements for a unique pair to obtain a set of coefficients characterizing said signal at said unique pair.

9. The method of claim 8 wherein said step of determining a location vector comprises solving a set of non-linear equations comprising said coefficients.

10. The method of claim 6 wherein said step of measuring further comprises measuring during two or more periods of time.

11. The method of claim 10 wherein the step of determining a location vector further comprises fitting said plurality of sample measurements from said two or more periods of time for a unique pair to obtain a set of coefficients characterizing said signal at said unique pair.

12. The method of claim 11 wherein said step of determining a location vector comprises solving a set of non-linear equations comprising said coefficients.

13. The method of claim 1 further comprising selecting a plurality of unique pairs of signal generating electrodes and repeating said step of measuring said signal for each unique pair of signal generating electrodes.

14. The method of claim 13 wherein all electrodes are used as signal-generating electrodes.

15. The method of claim 13 wherein said set of at least five electrodes comprises at least six electrodes and less than all electrodes are used as signal generating electrodes.

16. The method of claim 15 wherein said set of at least six electrodes comprises six or seven electrodes and at least three electrodes are used as signal generating electrodes.

17. The method of claim 1 wherein said chamber comprises a first region filled with blood or body fluids having a first set of dielectric properties and a chamber wall comprising a second region having a second set of dielectric properties.

18. The method of claim 17 further comprising generating said signal in a range where the dielectric properties of said first and second regions are substantially equivalent.

19. The method of claim 17 further comprising
selecting a set of test frequencies,
determining location vectors for each electrode using at least two frequencies,
finding a difference between location vectors determined using one frequency and location vectors using another frequency,
selecting one of said location vectors when said difference is less than a pre-selected value, and
determining a new location vector using a further frequency from said set of test frequencies when said difference is greater than said pre-selected value.

20. The method of claim 19 further comprising
projecting said location vectors as a function of frequency to a frequency range where said first and second regions have substantially equivalent dielectric properties and
determining a projected location vector at a frequency in said frequency range.

21. The method of claim 17 further comprising
selecting a set of test frequencies,
determining location vectors for each electrode using at at least some of frequencies,
projecting said location vectors as a function of frequency to a frequency range where said first and second regions have substantially equivalent dielectric properties, and
determining a projected location vector at a frequency in said frequency range.

22. An apparatus for determining a spatial relationship between a plurality of electrodes implanted in a human body, said apparatus comprising
a set of at least five electrodes mounted on at least one lead and implanted in a chamber in the body, a first one of said two electrodes being an origin and said first and second electrodes defining a first axis; and a third electrode non-colinear with said first and second electrodes, said third electrode defining a second axis,
a signal generator producing a periodic signal having a frequency, said signal generator being selectively connected between two signal-generating electrodes,
a voltage measuring device, said signal measuring device being selectively connected to pairs of electrodes taken from said set of electrodes, excluding said two signal generating electrodes; and
means for determining a location vector for each electrode from said measured signals.

23. The apparatus of claim 22 wherein said first and second electrodes are separated by a known distance.

24. The apparatus of claim 22 further comprising means for measuring at every electrode at least once.

25. The apparatus of claim 22 further comprising means for determining a location vector for each real electrode and means for determining a location vector for a virtual electrode associated with each real electrode.

26. The apparatus of claim 25 further comprising means for determining the location of a dielectric boundary between a real electrode and a virtual electrode associated with said real electrode.

27. The apparatus of claim 1 further comprising means for acquiring a plurality of sample measurements for each unique pair during a period of time when said signal is generated between said signal-generating electrodes.

28. The apparatus of claim 27 further comprising a digital computer for solving a set of non-linear equations to determine a location vector.

29. The apparatus of claim 27 further comprising means for determining a location vector further comprises fitting said plurality of sample measurements for a unique pair to obtain a set of coefficients characterizing said signal at said unique pair.

30. The apparatus of claim 29 further comprising means for solving a set of non-linear equations comprising said coefficients.

31. The apparatus of claim 27 wherein said means for measuring further comprises means for measuring during two or more periods of time.

32. The apparatus of claim 31 further comprising means for fitting said plurality of sample measurements from said two or more periods of time for a unique pair to obtain a set of coefficients characterizing said signal at said unique pair.

33. The apparatus of claim 32 further comprising means for solving a set of non-linear equations comprising said coefficients.

34. The apparatus of claim 22 further comprising means for selecting a plurality of unique pairs of signal generating electrodes and means for repeatedly measuring said signal for each unique pair of signal generating electrodes.

35. The apparatus of claim 34 wherein all electrodes are used connectable to said signal generator.

36. The apparatus of claim 34 comprising at least six electrodes and less than all electrodes are connected to said signal generator.

37. The apparatus of claim 22 wherein said dielectric medium comprises a first region having a first set of dielectric properties and a second region having a second set of dielectric properties.

38. The apparatus of claim 37 wherein said signal generator can generate signals in a range where the dielectric properties of said first and second regions are substantially equivalent.

39. The apparatus of claim 38 wherein said signal generator can generate signals in excess of 100 MHz.

40. The apparatus of claim 37 further comprising
means for selecting a set of test frequencies,
means for finding a difference between location vectors determined using one frequency and location vectors using another frequency, and
means for selecting one of said location vectors when said difference is less than a pre-selected value.

41. The apparatus of claim 40 further comprising
means for projecting said location vectors as a function of frequency to a frequency range where said first and second regions have substantially equivalent dielectric properties.

42. The apparatus of claim 31 further comprising
means for selecting a set of test frequencies, and
means for projecting location vectors as a function of frequency to a frequency range where said first and second regions have substantially equivalent dielectric properties.

43. A method for determining a spatial relationship between a plurality of electrodes in a dielectric medium comprising
placing a set of at least five electrodes in a dielectric medium;
designating a first electrode as an origin;
designating a direction from said first electrode to a second electrode as a first axis,
selecting a third electrode, said third electrode being non-colinear with said first and second electrodes, said third electrode defining a second axis,
defining a third axis related to said first and second axes;
generating a periodic signal having a frequency between two signal-generating electrodes,
measuring said signal at pairs of electrodes taken from said set of electrodes, excluding said two signal generating electrodes; and
determining a location vector for each electrode from said measured signals.

44. An apparatus for determining a spatial relationship between a plurality of electrodes in a dielectric medium comprising
a set of at least five electrodes in a dielectric medium,
a first electrode defining an origin and a second electrode defining a first axis from said origin;
a third electrode, said third electrode being non-colinear with said first and second electrodes, said third electrode defining a second axis,
a signal generator producing a periodic signal having a frequency, said signal generator being selectively connected between two signal-generating electrodes,
a voltage measuring device, said signal measuring device being selectively connected to pairs of electrodes taken from said set of electrodes, excluding said two signal-generating electrodes; and
means for determining a location vector for each electrode from said measured signals.
